(19) United States
(12) Patent Application Publication
(10) Pub. No.: US 2026/0339853 A1
(43) Pub. Date: Oct. 29, 2026

(54) SECURE HARDWARE CRYPTOWALLETS FOR SMARTPHONES

(71) Applicant: Thirdwayv, Inc., Irvine, CA (US)

(72) Inventors: Nabil Wasily, Foothill Ranch, CA (US); Michael Atef Ayoub, Irvine, CA (US)

(73) Assignee: Thirdwayv, Inc., Irvine, CA (US)

(21) Appl. No.: 19/099,177

(22) PCT Filed: Sep. 26, 2023
(86) PCT No.: PCT/US2023/033750

§ 371 Date: Jan. 28, 2025

Related U.S. Application Data
(60) Provisional application No. 63/410,575, filed on Sep. 27, 2022.

(51) Int. Cl. G06F 13/12; G06Q 20/36

(52) U.S. Cl. CPC G06F 13/12; G06Q 20/36; G06Q 2220/00

(57) ABSTRACT

Cryptocurrency transactions are increasing in prevalence. An important security practice for cryptocurrency transactions includes maintaining a cryptowallet with a private key for signing cryptocurrency transactions. Hardware based cryptowallets with a secure element retaining the private key are highly secure. A cable assembly connecting a smartphone baseband processor to a display screen of a smartphone may have a hardware based cryptowallet disposed in the cable assembly. This cable assembly may have a secure element that connects to the smartphone baseband processor via a wireless connection. In this manner, the display screen may be utilized by both the smartphone baseband processor and the secure element, and private keys may be safely retained in the secure element so that cryptocurrency transactions may be performed by an application running on the smart phone but without exposing the private keys to the smartphone baseband processor.

### CROSS-REFERENCE TO RELATED APPLICATIONS

This application is based upon and claims the benefit and priority to U.S. Provisional Patent Application No. 63/410,575, entitled “SECURE HARDWARE CRYPTO WALLETS FOR SMARTPHONES,” filed on Sep. 27, 2022, the entire content of which is incorporated herein by reference.

### FIELD

The present invention is directed to cryptowallets, and more specifically, the present disclosure is directed to secure hardware for cryptowallets.

### BACKGROUND

Cryptowallets are used to generate and store the cryptographic keys required for performing cryptocurrency transactions. The private keys of a cryptowallet are usually generated via a deterministic hierarchical scheme based on one master secret, called the seed value. In addition to a cryptographic seed value, a private key and/or a wallet personal-identification-number (PIN) code are also important to maintain securely. If the seed value, private key, and/or wallet PIN code are leaked or compromised, a hacker may gain control of the cryptowallet and/or initiate unwanted transactions.

### SUMMARY

A hardware cryptowallet cable assembly is disclosed. The hardware cryptowallet cable assembly is for connecting a smartphone baseband processor to a display screen. The cable assembly may include a first connector connectable to the smartphone baseband processor. The cable assembly may include a second connector connectable to the display screen. The cable assembly may include a cable interconnecting the first connector and the second connector. The cable assembly may include a hardware cryptowallet disposed in at least one of (1) the first connector, (2) the second connector, and/or (3) the cable.

One or more further feature or aspect may be provided. For instance, the hardware cryptowallet may include a secure element wirelessly connectable to the smartphone baseband processor via a communication interface. The communication interface may be a Bluetooth low energy (BLE) transceiver. The communication interface may be a wireless transceiver. The hardware cryptowallet may include a multiplexer configured to selectably connect the display screen to (i) the secure clement and/or (ii) the smartphone baseband processor, whereby the secure element may display transaction details of a cryptocurrency transaction on the display screen. The hardware cryptowallet may also have sensor connected to the secure element to receive biometric data from a user to verify an identity of a user by the secure element.

The hardware cryptowallet cable assembly may be a direct replacement for a cable connecting the smartphone baseband processor and the display screen, wherein the hardware cryptowallet cable assembly interacts with an application running on the smartphone baseband processor via a wireless connection of the hardware cryptowallet to the smartphone baseband processor. The hardware cryptowallet cable assembly may replace the cable without any other changes being made to the smartphone baseband processor and the display screen.

A smartphone having a hardware cryptowallet is disclosed. The smartphone may include a smartphone baseband processor configured to run smartphone applications. The smartphone may include a display screen configured to display user-readable output from the smartphone baseband processor. The smartphone may include a cable assembly connecting the smartphone baseband processor to the display screen. The cable assembly may have a hardware cryptowallet.

One or more further feature or aspect may be provided. For instance, the cable assembly may include a first connector connected to the smartphone baseband processor, a second connector connected to the display screen, and a cable interconnecting the first connector and the second connector. The hardware cryptowallet may be disposed in at least one of (1) the first connector, (2) the second connector, and (3) the cable. The hardware cryptowallet may have a secure element wirelessly connected to the smartphone baseband processor via a communication interface. The communication interface may be a Bluetooth low energy (BLE) transceiver. The communication interface may be a wireless transceiver. The hardware cryptowallet may include a multiplexer configured to selectably connect the display screen to (i) the secure element and (ii) the smartphone baseband processor, whereby the secure element may display transaction details of a cryptocurrency transaction on the display screen. The hardware cryptowallet may include a sensor connected to the secure element to receive biometric data from a user to verify an identity of the user by the secure element.

The hardware cryptowallet cable assembly may be a direct replacement for a cable connecting the smartphone baseband processor and the display screen, wherein the hardware cryptowallet cable assembly interacts with an application running on the smartphone baseband processor via a wireless connection of the hardware cryptowallet to the smartphone baseband processor. The hardware cryptowallet cable assembly may replace the cable without any other changes being made to the smartphone baseband processor and the display screen.

A method of performing a cryptocurrency transaction with a smartphone having a hardware cryptowallet is provided. The method may include sending, by a smartphone application running on a smartphone baseband processor of the smartphone, a cryptocurrency transaction to the hardware cryptowallet via a wireless communication interface. The method may include receiving, by a secure element of the hardware cryptowallet, the transaction, parsing the transaction, and generating a visible representation of the transaction. The method may include sending, by the secure element, the visible representation to a controller to frame buffer the visible representation. The method may include providing, by the secure element, a control signal to a multiplexer connected to (i) the smartphone baseband processor and to (ii) the controller, the control signal directing the multiplexer to (a) disconnect the smartphone baseband processor from the display screen and to (b) connect the secure element to the display screen. The method may include receiving an interaction from a user by a sensor of the hardware cryptowallet, the interaction verifying the accuracy of the visible representation. The method may include signing, by the secure element, the transaction with a private key in response to the interaction to generate a signed transaction. The method may include transmitting, by the secure element, the signed transaction to the smartphone application running on the smartphone baseband processor via the wireless communication interface.

The method may include one or more other aspects. For instance, the visible representation may be a transaction amount and a receiver address. The hardware cryptowallet may be in a cable assembly connecting the smartphone baseband processor of the smartphone to a display screen of the smartphone.

### BRIEF DESCRIPTION OF THE FIGURES

A more complete understanding of the present invention may be derived by referring to the detailed description and claims when considered in connection with the Figures, where like reference numbers refer to similar elements throughout the Figures, and:

FIG. 1 is a diagram of components of a smartphone, in accordance with various embodiments;

FIG. 2 is a diagram of components of a smartphone having a secure hardware cryptowallet, in accordance with various embodiments;

FIGS. 3A-3C are diagrams of different hardware cryptowallet cable assemblies for a secure hardware cryptowallet, in accordance with various embodiments;

FIG. 4 is a detailed illustration of components of a secure hardware cryptowallet for a smartphone, in accordance with various embodiments; and

FIG. 5 depicts a method of performing a cryptocurrency transaction with a smartphone having a secure hardware cryptowallet, in accordance with various embodiments.

### DETAILED DESCRIPTION

The following description is of various exemplary embodiments only, and is not intended to limit the scope, applicability, or configuration of the present disclosure in any way. Rather, the following description is intended to provide a convenient illustration for implementing various embodiments including the best mode. As will become apparent, various changes may be made in the function and arrangement of the elements described in these embodiments without departing from the scope of the appended claims.

Cryptowallets are used to generate and store cryptographic keys required for performing cryptocurrency transactions. Cryptowallets include at least two common varieties. Software wallets are cryptowallets that are a smartphone or desktop application that stores the cryptographic keys and also manages incoming and outgoing transactions. Hardware wallets are cryptowallets that have an embedded device, such as a secure element (SE), that stores the private keys used for signing outgoing transactions. The hardware wallet communicates with a smartphone, desktop, or web application “watch-only wallet” that only stores the corresponding public keys of the hardware wallet's private keys. A watch-only wallet monitors incoming transactions and prepares unsigned outgoing transactions for the embedded device but cannot sign outgoing transactions. The embedded device verifies the transaction details, retrieves the applicable signing key, signs the transaction, and sends the signed transaction back to the smartphone, desktop, or web application to be broadcast to mining nodes.

Hardware cryptowallets are known to be more secure than software cryptowallets because the software cryptowallets coexist with other applications on a smartphone or desktop and thus the private keys are susceptible to software attacks on the shared platform, while the keys in a hardware cryptowallet never leave the embedded device. Access to hardware cryptowallet operations is often protected by a personal identification number (PIN) or sensed biometric such as a fingerprint or facial recognition.

Private keys of a cryptowallet may be generated via a deterministic hierarchical scheme based on one master secret. The master secret is called the seed value. This seed value is important and must be maintained securely. The private keys must also be maintained securely. Moreover, a user may, in some instances, be required to enter a PIN code associated with the cryptowallet to authorize various transactions. The PIN code should also be maintained securely.

In parallel with these security requirements, wallet users also desire operational convenience. One way to increase convenience is to perform cryptowallet transactions on a smartphone. However, while a smartphone increases convenience, it also introduces security risks.

Thus, performing cryptowallet transactions on a smartphone is difficult for various reasons. For instance, smartphone software is built on an operating system (for example, iOS or Android) that controls and oversees the operation of applications such as wallet software applications. Malware or malicious software can compromise the operation of applications and/or operating systems. Malicious firmware can compromise the operation and security of smartphone storage components such as electronic memory and user interface (UI) components such as displays and touch controllers.

These attacks can lead to compromise of user input and output to/from the smartphone UI as well as storage of wallet secrets in a smartphone electronic memory. Such compromise may lead to a malicious attacker gaining access to the cryptowallet private key, seed phrase, and/or wallet PIN code. An attacker could compromise a user transaction input or output, permitting an attacker to change a cryptocurrency transaction recipient or a cryptocurrency transaction amount.

This disclosure includes systems and methods to address both convenience concerns and security concerns, while avoiding costly or difficult changes to a smartphone hardware. This disclosure provides a hardware solution that isolates cryptowallet operations. This disclosure preserves the current form-factor of a smartphone (for instance, a phone having a single touchscreen). This disclosure enables smartphone manufacturers to integrate a hardware cryptowallet into a smartphone with minimal changes to existing smartphone hardware and software. This disclosure enables a hardware cryptowallet to be added via an add-on module, or via integration into a cable such as a cable or a connector connecting the smartphone screen to other components of the smartphone. By integrating a hardware cryptowallet into a cable or connector connecting the screen to other components of the smartphone, a hardware cryptowallet may be added without requiring any changes to the hardware or software of the smartphone except that rather than assembling the phone with a traditional cable or connector connecting the screen to the other components, instead a cable or connector with an embedded hardware cryptowallet is used.

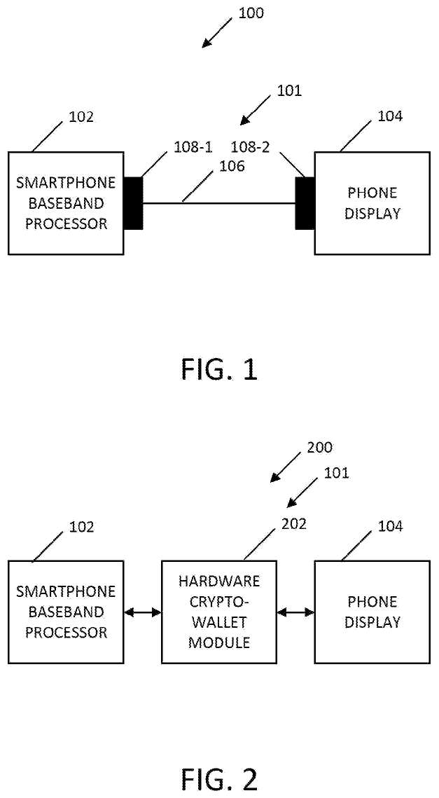

With reference to FIG. 1, a smartphone 100 is depicted in block diagram form. The smartphone 100 has a smartphone baseband processor 102. As used herein, the smartphone baseband processor 102 comprises the circuitry and other components making up the smartphone 100. The smartphone 100 also has a phone display 104. The phone display 104 comprises the user-interface components that permit human interaction with the features of the smartphone baseband processor 102. For example, the phone display 104 may be a liquid-crystal display (LCD) display. The phone display 104 may have a touch sensor, such as a digitizer or other mechanism by which a user may provide inputs to the phone display 104, such as by clicking, typing on an on-screen keyboard, drawing, writing, and/or the like.

The smartphone baseband processor 102 and the phone display 104 are connected by a cable assembly 101. The cable assembly 101 may include a cable 106. The cable assembly 101 may include a first connector 108-1. The first connector 108-1 may be disposed at one end of the cable 106. The first connector 108-1 may connect the cable 106 at the one end to the smartphone baseband processor 102. The cable assembly 101 may have a second connector 108-2. The second connector 108-2 may connect the cable 106 at the other end to the phone display 104.

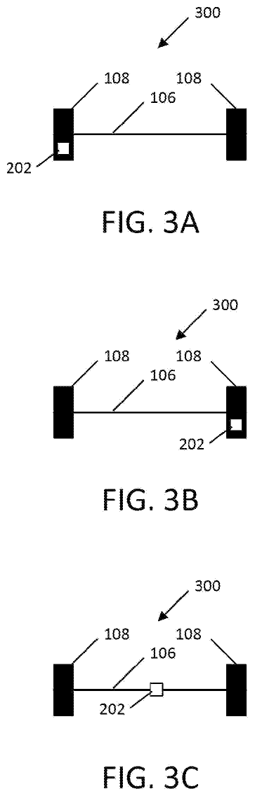

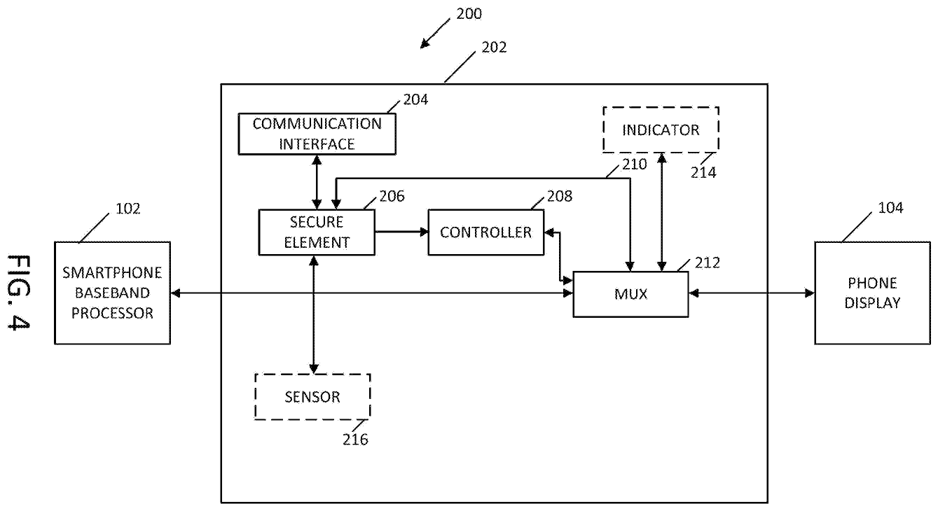

Turning to FIG. 2, in one example, a smartphone 200 having a secure hardware cryptowallet is depicted. The smartphone 200 may include a smartphone baseband processor 102 and a phone display 104. A hardware cryptowallet module 202 may be disposed in electrical connection between the smartphone baseband processor 102 and the phone display 104. This hardware cryptowallet module 202 may be disposed in the cable assembly 101. The combination of a cable assembly 101 and a hardware cryptowallet module 202 may be a hardware cryptowallet cable assembly 300 (FIGS. 3A-3C). For instance, with reference to FIGS. 3A-3C, various different configurations of a hardware cryptowallet module 202 are illustrated disposed in various aspects of the cable assembly 101.

In FIG. 3A, a hardware cryptowallet cable assembly 300 is depicted. A hardware cryptowallet cable assembly 300 includes a cable 106 with a first connector 108-1 at one end and a second connector 108-2 at the other end. A hardware cryptowallet module 202 is disposed in the first connector 108-1.

In FIG. 3B, a hardware cryptowallet cable assembly 300 is depicted. A hardware cryptowallet cable assembly 300 includes a cable 106 with a first connector 108-1 at one end and a second connector 108-2 at the other end. A hardware cryptowallet module 202 is disposed in the second connector 108-2.

In FIG. 3C, a hardware cryptowallet cable assembly 300 is depicted. A hardware cryptowallet cable assembly 300 includes a cable 106 with a first connector 108-1 at one end and a second connector 108-2 at the other end. A hardware cryptowallet module 202 is disposed in the cable 106.

Thus one may appreciate that a smartphone 100 (FIG. 1) may be assembled with conventional components, but rather than a cable assembly 101 (FIG. 1) to connect the smartphone baseband processor 102 (FIG. 1) and the phone display 104 (FIG. 1), a hardware cryptowallet cable assembly 300 (FIGS. 3A-3C) is substituted for the cable assembly 101 (FIG. 1), adding hardware cryptowallet functionality to the smartphone 100 (FIG. 1).

Turning now to FIG. 4, an example hardware cryptowallet module 202 that is installed in a smartphone 200 is shown in detail. The hardware cryptowallet module 202 may comprise a variety of different components. For example, the hardware cryptowallet module 202 may include a communication interface 204. In various embodiments, the communication interface 204 may be a Bluetooth low energy communication transceiver. In further embodiments, the communication interface 204 may be a serial communication transceiver. The communication interface 204 may be a wireless communication interface or a wired communication interface. In various embodiments, the communication interface 204 is a wireless communication interface configured to communicate with a corresponding interface of the smartphone baseband processor 102 without wired connection. In this manner, the hardware cryptowallet module 202 may communicate with the smartphone baseband processor 102 of the smartphone 200 without requiring any changes to the smartphone baseband processor 102. In this manner, the use of a hardware cryptowallet cable assembly 300 (FIGS. 3A-3C) retrofits a smartphone to have a hardware cryptowallet module 202 without changing the smartphone baseband processor 102 architecture.

The hardware cryptowallet module 202 may include a secure element 206. A secure element 206. A secure element (SE) is a tamper-resistant platform capable of securely hosting applications and their confidential and cryptograph data. For instance, a secure element 206 may have a memory and or processor for storage of seeds, or private keys and for execution of cryptographic operations with seeds or private keys. For instance, one or more of key pairs may be generated within the SE, for enhanced security, such that the key is hardware-backed and cannot be retrieved outside a SE. In various instances, one or more of the private keys are retained in a SE. This also facilitates improved security. By retaining the key within the SE, the key is secured from retrieval outside the SE, but may still be utilized for cryptographic operations. The secure element may store a private key (seed phrase), parse a cryptocurrency transaction, generate visible representation of the transaction to be presented to a user, and/or receive user confirmation via a push-button or other input connected to secure element.

The hardware cryptowallet module 202 may have a controller 208. The controller 208 may be a microprocessor. In further instances, the controller 208 is a frame buffer. The secure element 206 may generate human readable outputs for display on a phone display 104 and the controller 208 may buffer these outputs so that they are properly renderable by the phone display 104. The frame buffer may be a video frame buffer and/or an image frame buffer. The frame buffer receives a visible representation of a transaction for presentation to a phone user. In various embodiments, the frame buffer is loaded from the secure element via a direct serial interface. The frame buffer could also be part of a microprocessor that receives the transaction visible representation. Thus, the controller 208 converts a visible transaction representation to a video/image stream over a display interface (for example MIPI DSI).

The hardware cryptowallet module 202 may include a display interface multiplexer (MUX) 212. The MUX 212 is a device that is operable to select between multiple inputs and provide the selected one of the multiple inputs on an output. By using a MUX 212, a source of data provided to a phone display 104 may be switched. In this manner, the phone display 104 may be made to display data provided by the smartphone baseband processor 102 of the smartphone 200 or may be made to display data provided by a different device, such as the secure element 206. The MUX 212 is connected to the smartphone baseband processor 102 and is also connected to the secure element 206. The MUX 212 is controlled via a control line 210 that is also connected to the secure element 206. In this manner, the secure element 206 may operate the MUX 212 to switch the data provided to the phone display 104 from that of the smartphone baseband processor 102 to that of the secure element 206 so that a user may interact securely with the secure element 206. Stated differently, the MUX 212 multiplexes the phone display 104 between the smartphone baseband processor 102 and the secure element 206 in response to a control signal from the secure element 206.

The hardware cryptowallet module 202 may include optional components. For instance, an indicator 214 may be provided. The indicator 214 may comprise a light-emitting diode. The indicator 214 may comprise another human-readable indicator device, such as a haptic feedback device. The indicator 214 is connected to the MUX 212 and operates to indicate which input is presently being provided to the phone display 104. For instance, the indicator 214 may illuminate in response to the MUX 212 connecting the secure element 206 to the phone display 104.

Continuing the discussion of optional components, a sensor 216 may also be provided. The sensor 216 may be controlled directly by the secure element 206. The sensor 216 may operate to sense biometric data. For instance, the sensor 216 may be a touch sensor to detect a fingerprint or other unique physiological feature of a user. The sensor 216 may be an optical sensor to detect features of a user's face or other unique physiological features of a user. In various embodiments, the sensor 216 is not directly connected to the secure element 206 but is a component of a phone display 104 such as a touch screen digitizer, or existing. sensor of the smartphone 200. The secure element 206 may use the sensor 216 to verify an identity of a user interacting with the secure element 206. In this manner, unauthorized use of the secure element 206 may be prevented. This further reduces a risk that a PIN code or other information be leaked. In various embodiments, the sensor 216 is a button that is pressed by a user. In further embodiments, the sensor 216 is a touch screen aspect of the phone display 104 and receives entry by a user of a PIN code.

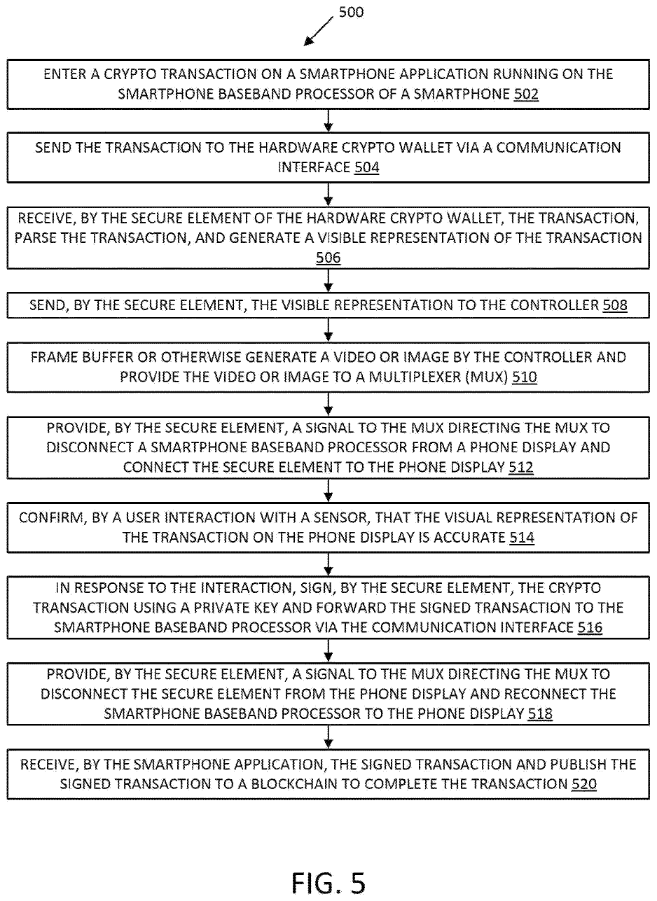

Having discussed each aspect of the hardware cryptowallet module 202, attention is now directed to FIG. 5 for a discussion of an example method of using a hardware cryptowallet module 202. In various embodiments, a method 500 may include a user entering a cryptocurrency transaction on a smartphone application running on the smartphone baseband processor of a smartphone (block 502). The smartphone application may send the transaction to the hardware cryptowallet via the communication interface (block 504). The secure element of the hardware cryptowallet receives the transaction, parses the transaction, and generates a visible representation of the transaction (block 506). In various embodiments, the visible representation comprises a transaction amount and a receiver address. The secure clement sends the visible representation to the controller (block 508). The controller may frame buffer or otherwise generate a video or image and provide the video or image to the MUX (block 510). The secure element may also provide a signal to the MUX directing the MUX to disconnect the smartphone baseband processor from the phone display and connect the secure element to the phone display (block 512). The user may interact with the sensor to confirm that the visual representation of the transaction on the phone display is accurate (block 514). This interaction may take various forms. For instance, a user may press a button. The user may enter a PIN code on the phone display 104 via a touch screen of the phone display 104. The user may provide biometric information such touching the sensor to confirm a fingerprint or looking at the sensor to confirm facial recognition. In response to the interaction, the transaction is confirmed as accurate, and in various embodiments, the identity of the user is confirmed as authorized. Thus, in response to the interaction, the secure element may sign the cryptotransaction using a private key and forward the signed transaction to the smartphone baseband processor via the communication interface (block 516). The secure element may provide a signal to the MUX directing the MUX to disconnect the secure element from the phone display and reconnect the smartphone baseband processor to the phone display (block 518). The smartphone application receives the signed transaction and publishes the signed transaction to a blockchain to complete a transaction (block 520).

The present disclosure has been described with reference to various embodiments. However, one of ordinary skill in the art appreciates that various modifications and changes can be made without departing from the scope of the present disclosure. For example, features of different embodiments may be combined. Accordingly, the specification is to be regarded in an illustrative rather than a restrictive sense, and all such modifications are intended to be included within the scope of the present disclosure.

For the sake of brevity, conventional techniques for manufacturing and construction may not be described in detail herein. Furthermore, the connecting lines shown in various figures contained herein are intended to represent exemplary functional relationships and/or physical couplings between various elements. It should be noted that many alternative or additional functional relationships or physical connections may be present in a practical method of construction. As used herein, mechanical communication means any joint, connection, bond, or arrangement whereby an article is held, retained, or fixed in relatively static spatial relationship to another article. As used herein, electronic communication means any wired, wireless, analog, digital, or other mechanism whereby information is communicated between machines, circuits, or devices.

Benefits, other advantages, and solutions to problems have been described herein with regard to various embodiments. However, the benefits, advantages, solutions to problems, and any elements that may cause any benefit, advantage, or solution to occur or become more pronounced are not to be construed as critical, required, or essential features or elements of the invention. Moreover, where a phrase similar to “at least one of A, B, and C” or “at least one of A, B, or C” is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

As used herein, the singular forms “a,” “an,” and “the” include plural referents unless the context clearly dictates otherwise. As used herein, the terms “for example,” “for instance,” “such as,” or “including” are meant to introduce examples that further clarify more general subject matter. Unless otherwise specified, these examples are embodiments of the present disclosure, and are not meant to be limiting in any fashion.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f), unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A hardware cryptowallet cable assembly for connecting a smartphone baseband processor to a display screen, the cable assembly comprising:
a first connector connectable to the smartphone baseband processor;
a second connector connectable to the display screen;
a cable interconnecting the first connector and the second connector; and
a hardware cryptowallet disposed in at least one of (1) the first connector, (2) the second connector, and/or (3) the cable.

2. The hardware cryptowallet cable assembly of claim 1, wherein the hardware cryptowallet comprises a secure element wirelessly connectable to the smartphone baseband processor via a communication interface.

3. The hardware cryptowallet cable assembly of claim 2, wherein the communication interface comprises a Bluetooth low energy (BLE) transceiver.

4. The hardware cryptowallet cable assembly of claim 2, wherein the communication interface comprises a wireless transceiver.

5. The hardware cryptowallet cable assembly of claim 2, wherein the hardware cryptowallet comprises a multiplexer configured to selectably connect the display screen to (i) the secure element and (ii) the smartphone baseband processor, whereby the secure element may display transaction details of a cryptocurrency transaction on the display screen.

6. The hardware cryptowallet cable assembly of claim 1, wherein the hardware cryptowallet cable assembly is a direct replacement for a cable connecting the smartphone baseband processor and the display screen, wherein the hardware cryptowallet cable assembly interacts with an application running on the smartphone baseband processor via a. wireless connection of the hardware cryptowallet to the smartphone baseband processor.

7. The hardware cryptowallet cable assembly of claim 6, wherein the hardware cryptowallet cable assembly replaces the cable without any other changes being made to the smartphone baseband processor and the display screen.

8. The hardware cryptowallet cable assembly of claim 2, wherein the hardware cryptowallet further comprises a sensor connected to the secure element to receive biometric data from a user to verify an identity of a user by the secure element.

9. A smartphone having a hardware cryptowallet, the smartphone comprising:
a smartphone baseband processor configured to run a smartphone application;
a display screen configured to display user-readable output from the smartphone application on the smartphone baseband processor; and
a cable assembly connecting the smartphone baseband processor to the display screen,
wherein the cable assembly comprises a hardware cryptowallet.

10. The smartphone according to claim 9, wherein the cable assembly further comprises:
a first connector connected to the smartphone baseband processor;
a second connector connected to the display screen; and
a cable interconnecting the first connector and the second connector,
wherein the hardware cryptowallet is disposed in at least one of (1) the first connector, (2) the second connector, and (3) the cable.

11. The smartphone according to claim 10, wherein the hardware cryptowallet comprises a secure element wirelessly connected to the smartphone baseband processor via a communication interface.

12. The smartphone according to claim 11, wherein the communication interface comprises a Bluetooth low energy (BLE) transceiver.

13. The smartphone according to claim 10, wherein the communication interface comprises a wireless transceiver.

14. The smartphone according to claim 11, wherein the hardware cryptowallet comprises a multiplexer configured to selectably connect the display screen to (i) the secure element and (ii) the smartphone baseband processor, whereby the secure element may display transaction details of a cryptocurrency transaction on the display screen.

15. The smartphone according to claim 10, wherein the hardware cryptowallet cable assembly is a direct replacement for a cable connecting the smartphone baseband processor and the display screen, wherein the hardware cryptowallet cable assembly interacts with the smartphone application running on the smartphone baseband processor via a wireless connection of the hardware cryptowallet to the smartphone baseband processor.

16. The smartphone according to claim 15, wherein the hardware cryptowallet cable assembly replaces the cable without any other changes being made to the smartphone baseband processor and the display screen.

17. The smartphone according to claim 11, wherein the hardware cryptowallet further comprises a sensor connected to the secure element to receive biometric data from a user to verify an identity of the user by the secure element.

18. A method of performing a cryptocurrency transaction with a smartphone having a hardware cryptowallet, the method comprising:
sending, by a smartphone application running on a smartphone baseband processor of the smartphone, a cryptocurrency transaction to the hardware cryptowallet via a wireless communication interface;
receiving, by a secure element of the hardware cryptowallet, the transaction, parsing the transaction, and generating a visible representation of the transaction;
sending, by the secure element, the visible representation to a controller to frame buffer the visible representation;
providing, by the secure element, a control signal to a multiplexer connected to (i) the smartphone baseband processor and to (ii) the controller, the control signal directing the multiplexer to (a) disconnect the smartphone baseband processor from the display screen and to (b) connect the secure element to the display screen;
receiving an interaction from a user by a sensor of the hardware cryptowallet, the interaction verifying the accuracy of the visible representation;
signing, by the secure element, the transaction with a private key in response to the interaction to generate a signed transaction; and
transmitting, by the secure element, the signed transaction to the smartphone application running on the smartphone baseband processor via the wireless communication interface.

19. The method according to claim 18, wherein the visible representation comprises a transaction amount and a receiver address.

20. The method according to claim 18, wherein the hardware cryptowallet is in a cable assembly connecting the smartphone baseband processor of the smartphone to a display screen of the smartphone.
